(19) United States
(12) Patent Application Publication
(10) Pub. No.: US 2009/0005648 A1
(43) Pub. Date: Jan. 1, 2009

(54) Method for the Diagnosis of Attention-Deficit/Hyperactivity Disorder

(76) Inventors: Martin H. Teicher, Rye, NH (US); Ann Polcari, Melrose, MA (US)

Correspondence Address: CLARK & ELBING LLP, 101 FEDERAL STREET, BOSTON, MA, 02110, US

(21) Appl. No.: 11/661,876

(22) PCT Filed: Sep. 2, 2005
(86) PCT No.: PCT/US05/31423

§ 371 Date: Jul. 28, 2008

Related U.S. Application Data
(60) Provisional application No. 60/606,627, filed on Sep. 2, 2004.

(51) Int. Cl. A61B 5/00

(52) U.S. Cl. 600/300

(57) ABSTRACT

The invention provides methods for determining the attentional state of a subject. These methods are useful for diagnosing subjects with a psychological or behavioral disorder. The invention also features methods for determining the effect of a therapy on the attentional state of a subject.

### BACKGROUND OF THE INVENTION

Attention Deficit/Hyperactivity Disorder (ADHD) is the most common neurobehavioral disorder of childhood, affecting between 4% and 12% of school aged children. Approximately $3 billion is spent annually on behalf of children diagnosed with ADHD.

Contrary to the popular belief that children outgrow ADHD in adolescence, studies have shown that 80% of children with ADHD still exhibit symptoms in adolescence. Moreover, with only a 50% remission rate from childhood to adulthood, the prevalence of ADHD in the general adult population is also significant, with upwards of 10 million adults affected in North America (see Weiss and Murray, Can. Med. Assoc. J. 168: 715-22, 2003). Indeed, research into the neurobiological features of ADHD in adults has had a substantial impact on establishing the validity of this disorder. Functional MRI studies have shown increased activity in the frontal striatal networks in adults with ADHD relative to normal subjects and positron emission tomography studies have shown decreased frontal cortical activity in ADHD-affected adults.

Although many cases of ADHD are appropriately diagnosed and managed in childhood, many others are not diagnosed until adulthood. Adults with ADHD may face physicians who are unfamiliar with the subtleties of adult presentations of a classically childhood disorder, and child psychiatrists are often unwilling to take on adult patients.

The classical assessment process used for adults suspected of having ADHD is similar to that for children with the disorder. This process involves documenting current and past symptoms, establishing that the symptoms cause impairment, obtaining a developmental and psychiatric history, and performing a physical examination.

Recently, it is common practice that a continuous performance test (CPT) form part of the physical examination. A subject's visual attention can be tested by displaying a series of visual stimuli, to which the subject is instructed to respond. Typically, the stimuli are of two types, and the subject is instructed to respond to only one of them. Data are collected for each stimulus presented including the type of stimulus, whether or not the subject responded, and if so, how long the subject took to respond. The continuous performance attention test has been in use since the mid 50's (Rosvold et al., J. of Consulting and Clinical Psychology 20: 343-350, 1956), with computerized versions available in the 1970's (Greenberg, Psychopharmacol. Bull. 23: 279-82, 1987). The previous methods of analysis of the raw data generated from these methods have typically distilled the data into a few numbers which do not capture the subject's fluctuations in attention.

Another method for assessing the visual attention capabilities of a subject involves determining how long a particular visual stimulus must be present before a subject can detect it (U.S. Pat. No. 5,801,810). This method does not reveal the attentional state of the subject, rather, it requires the subject to be fully attentive.

Other reported methods determine a subject's intensity of focused attention, concentration, and/or interest by measuring signals naturally emanating from the brain (U.S. Pat. Nos. 5,983,129 and 5,377,100). These brainwaves vary across subjects and even within the same subject; thus, these methods do not provide a reliable, well-defined number for classifying attentional states.

A diagnostic assessment of psychological conditions can be made by conducting a sequence of continuous performance tests where information is recorded to reflect the number of target stimuli correctly identified, the number of target stimuli missed, the number of responses to non-target stimuli, the number of non-target stimuli correctly missed, and the final interstimulus interval (U.S. Pat. No. 5,940,801). Improvements made to this method for evaluating ADHD children by the incorporating into the evaluation an analysis of the test subject's movement patterns (see Teicher et al., J. Am. Acad. Child Adolesc. Psychiatry 35: 334-42, 1996). A version of such a testing system (the M-MAT, McLean Motion Attention Test, test system, McLean Hospital, Belmont, Mass.) is now commercially available.

Although effective for children, the use of this test in adults gives variable results as adults are more likely to recognize higher order test patterns or test features that are supplementary to task performance (e.g., the detection of a test subject's movements) and compensate accordingly in their responses and/or actions, thereby skewing the results of the test and leading to an inaccurate diagnosis.

### SUMMARY OF THE INVENTION

Accordingly, in a first aspect, the invention features a method of determining the attentional state of a subject that includes: tracking the movements of the subject; presenting to the subject a sequence of a predetermined number of stimuli over a predetermined period of time, where the sequence includes an array that includes target stimuli that vary in the duration of their presentation, preferably from 100 to 400 milliseconds (or from 100 to as much as 600, 700, 800, 900, 1,000, or 1,500 milliseconds); scoring the target stimuli responded to by the subject; and determining the attentional state of the subject based on an evaluation of the subject's movements and the scoring of target stimuli responded to.

By “target stimuli” are meant stimuli, including audio and visual stimuli, that a test subject has been instructed to respond to. The status of a stimulus as a target stimulus can be unconditional, meaning that a test subject's response to it is always required. Alternatively, a stimulus can be a “working memory stimulus,” whose status as a target stimulus is conditional, meaning that a response is required if the stimulus meets one or more predetermined conditions. In one example of a working memory stimulus, the stimulus functions as a target stimulus when following a first pre-defined symbol in the array, but not as a target stimulus when following a second pre-defined symbol in the array. Alternatively, a working memory stimulus can function as a target stimulus only when a predetermined number of array members separate it from a predetermined symbol presented in the array.

By incorporating working memory stimuli into the test, greater degrees of test complexity can be achieved than when only unconditional target stimuli are used. Such complexity can be useful in identifying impulsive or distracted states of tested subjects, particularly those subjects that are twelve years old or older. In such older subjects, even one missed response to a target stimulus (an error of omission) can be indicative of a distracted state. Likewise, in older subjects, just one incorrect positive response to a non-target stimulus (an error of commission) can be indicative of an impulsive state.

Accordingly, a second aspect of the invention employs working memory stimuli and features a method of determining the attentional state of a subject that includes: tracking the movements of the subject; presenting to the subject a sequence of a predetermined number of stimuli over a predetermined period of time, wherein the sequence includes an array that includes target stimuli and working memory stimuli; scoring the target stimuli responded to by the subject;

and determining the attentional state of the subject based on an evaluation of the subject's movements and the scoring of target stimuli responded to. As before in the first aspect of the invention, the length of time that any of the target stimuli are presented to the subject can vary, preferably in the range of from 100 to 400 milliseconds (or from 100 to as much as 600, 700, 800, 900, 1,000, or 1,500 milliseconds).

In one embodiment of the second aspect, the method can further include scoring the number of working memory stimuli functioning as target stimuli responded to by the subject. In another embodiment, the method further includes measuring a first time period required for the test subject to respond to one of the unconditional target stimuli and measuring a second time period required for the subject to respond to one of the working memory stimuli, with the scoring including comparing the first time period to the second time period. Delays (latency periods) in responding to working memory stimuli are also indicative of distracted behavior. Conversely, the rapid responses to working memory stimuli of non-target status (i.e., a false response) are indicative of impulsive behavior.

In a third aspect, the invention features a method of determining the effects of a therapy on the attentional state of a subject by using a method of either the first or second aspect of the invention at least once before and at least once after the subject has been administered the therapy. The before and after difference in attentional state(s) of the subject, as measured by the method, are then compared and the therapy is determined to have an effect when before and after attentional state differences are observed. In an embodiment of this aspect, this method can be used to determine a preferred therapy for the treatment of a psychological, neurological, or behavioral disorder.

For any of the methods of the invention, the method can further include scoring the target stimuli not responded to by the subject, scoring the non-target stimuli responded to by the subject, or scoring both the target stimuli not responded to by the subject and the non-target stimuli responded to by the subject. A test subject's response to stimuli can be by any method, such as for example, pressing a key or the spacebar of a keyboard in communication with a computer, clicking a mouse in communication with a computer, using a computer touchscreen, activating an electronic button in communication with a recording device, etc.

For any of the methods of the invention, the subject's movements can be tracked during a non-responsive interval or during a responsive interval after the presentation of one stimulus and before the presentation of the next stimulus in the array. An evaluation can then be based on a comparison of the movements during the non-responsive time period and during the responsive time period, and a determination of a hyperactivity-related disorder made based, at least in part, on this evaluation. A subject's movements can be tracked by a camera, preferably positioned in front of the subject. Positioning a camera in such a position is particularly well-suited for monitoring the motions of a subject's lower extremities (e.g., legs and feet) in addition to movements of the upper body and head.

For any of the methods of the invention, the stimuli, preferably 1, 2, 3, 4, 5, 6, 7, 8, 9, 10, or more, can be audio sounds or visual symbols, such as, for example, numbers, letters, shapes, or combinations thereof. The stimuli can be presented using a computer screen or a speaker, with the subject's responses recorded and stored in a computer. In one embodiment, the methods can include the presentation of stimuli and the gathering of information (e.g., a subject's movements and responses to stimuli) over a computer network. When such a network is employed, a subject's attentional state can be determined when the subject is at a location that is remote from that of the person who is administrating the test.

In methods that use audio stimuli, the sound can originate from one location or a multitude of locations. In the methods that use visual stimuli presented on a screen, the screen location of each stimulus in the array can change. For those methods that use audio and/or visual working memory stimuli, their location can used to determine whether such stimuli function as target stimuli.

For visual stimuli, the array can include masking symbols which alter the subject's visual image of the preceding stimulus target, making it difficult for a subject to focus on the retinal afterimage of any presented stimulus. Preferably, masking stimuli are presented for a time interval of between 100 and 2,000 milliseconds. In one example, the masking stimuli are presented for 200 milliseconds.

The methods of the invention can be used to assess both overall attention and impulsivity and can be repeated three or more times to determine the pattern of attentional states and the time spent in each state. These methods can also be used to diagnose or aid in the diagnosis of psychological, neurological, or behavioral disorders, such as depression, an anxiety disorder, schizophrenia, a drug addiction, an eating disorder, an attention deficit disorder, an attention deficit and hyperactivity disorder, hyperkinetic disorder, a learning disorder, Alzheimer's disease, dementia, epilepsy, stroke or traumatic brain injury. In addition, these methods can be used to identify those subjects at risk for a psychological, neurological, or behavioral disorder.

For pre-adolescent or adolescent subjects, the methods of the invention can be used for evaluating the subject's readiness for starting school or being promoted to the next grade level. For adults, the methods be used to assess the ability of a subject to function well in a job requiring attentional vigilance (e.g., an air traffic controller, a pilot, an emergency room doctor, a surgeon, a police officer, a military officer, or a fire-fighter).

### DETAILED DESCRIPTION

The invention features methods of assessing the attentional state of a subject and measuring the fluctuations in the attentional state of the subject. These methods include two components, a motion detection system and a test that simultaneously assesses the vigilance, impulsivity, and working memory capacity of the test subject.

### Motion Detection System

A motion detection system is used to track the movement of the head and/or lower extremities of the individual being tested. Movement patterns are analyzed using procedures described by Paulus and Geyer, Neuropsychopharmacology 7: 15-31, 1992 and Teicher et al., J. Am. Acad. Child Adolsec. Psychiatry 35: 334-342, 1996, which are based on the concept of microevents. A new microevent begins whenever the marker moves more than a predetermined distance (e.g., 1.0 mm or more) from the location of the previous microevent, and is defined by its position and duration. From the sequence of microevents, the mean locomotor path length can be calculated, along with two scaling exponents.

The first exponent, the spatial scaling exponent, is a measure of the complexity of the movement and is calculated by ascertaining the logarithmic rate of information decay at progressively lower levels of resolution. Conceptually, if a marker is still or moving in a straight line, no information is lost if the marker's position is sampled less frequently. The total distance traversed can still be calculated. On the other hand, if a marker is moving in a convoluted path, then less frequent sampling smooths out the route and underestimates the distance traveled. Spatial complexity corresponds to the concept of fractal dimensions and ranges from 1.0 (straight line movement) to 2.0 (complex, convoluted movement patterns).

The other exponent, known as the temporal scaling exponent, is calculated from the log-log relationship between the frequency of the microevents and their duration. For a two-process model in which a marker is either in motion or immobile, stochastic theory dictates that there will be a greater number of brief periods of immobility than long periods of immobility (though not necessarily a greater amount of time). The log-log relationship provides a robust measure of relative activity versus inactivity and indicates the degree to which a subject is moving in the environment.

Any video camera or other motion-sensing device capable of detecting the movements of the test subject can be used. For example, the motion analysis device can be an infrared motion analysis system (e.g., Qualisys Medical AB, Gothenburg, Sweden) that includes a high-resolution CCD infrared video camera, an infrared strobe, and a video processor that provides hardware analysis of the video signal and outputs data to a computer. Such infrared motion analysis systems are known in the art, and are specifically designed to detect and record the precise vertical and horizontal position of small, light-weight infrared reflective markers. These markers are attached to the subject at various points, such as the head, shoulders, arms, legs, and feet.

As the subject moves these portions of his or her body, the IR motion analysis system detects changes in the positions of the markers and relays this information to a computer. Successive marker coordinates can be stored in the computer and analyzed using commercially available software (e.g., M-MAT software, McLean Hospital, Belmont, Mass.). Desirably, the camera is positioned in front of the subject, who is preferably in a seated position. The camera is also desirably positioned in such a manner that it can capture movements of the reflective markers in three dimensions, including movements towards and away from the display device. The motion analysis device can also include a second camera that can be used in combination with the first camera to better differentiate three dimensional movement. Adults with ADHD can manifest hyperactivity solely through excess movement of their lower extremities while seated. Therefore, the first camera can be used to track the movement of the subject's legs and/or feet or a second camera can be used to track the movement of the subject's lower extremities while the first camera tracks upper body movements.

### Continuous Performance Test

A second component of the methods of the invention is a test that simultaneously assesses the vigilance, impulsivity, and working memory capacity of the subject, and can be of sufficient difficulty to challenge adults.

One way of assessing attention and reaction time is to provide a subject with a continuous performance test (“CPT”) and record the subject's performance. A typical CPT involves presenting the subject with a series of stimuli and instructing the subject to respond only to certain target stimuli. The subject's performance is scored based on the number of target stimuli correctly identified, the number of target stimuli missed, the number of responses to non-target stimuli, the number of non-target stimuli correctly missed, and the response time (see, for example, U.S. Pat. No. 5,940,801). CPTs of the invention typically have a duration of about 20 minutes, but can be for shorter or longer periods of time as warranted by the complexity of the test, the disorder that the subject is being tested for, or the age of the subject.

In one example, a subject's visual attention can be tested by displaying an array of visual stimuli on a computer screen, for which different responses are required of the subject. The stimuli can be any sort of visual image, including but not limited to, individual symbols, numbers, letters, or shapes, or a combination thereof. Typically, the test requires the subject to distinguish between similar visual images, such as a four pointed start, a five-pointed star, an eight-pointed star, and a sixteen-pointed star. For example, in a test that includes a working memory stimulus, the subject is instructed to press the space bar on the computer's keyboard if an eight-pointed star is displayed on the computer screen, and to do nothing when a five-pointed star appears on the screen. In addition, the subject is instructed to respond to a four-pointed star (the working memory stimulus) if it is preceded at any time by a four-pointed star without an intervening sixteen-pointed star. Data are collected for each individual image presentation, including the type of stimulus (e.g., four-pointed star, five-pointed star, or eight-pointed star), whether or not the subject responded, and, if so, the amount of time the subject took to respond. From this raw data, the percentage of correct responses to the target stimulus, percentage of correct passes to the non-target stimulus, average response time, response time variability, and other statistics may be obtained. At the end of the test, the recorded data (e.g., key press information and movement information) can be processed by the computer or transmitted over an Internet connection to a central processing station, where a report is generated and transmitted back to the testing site (see, for example, U.S. Application Ser. No. 60/243,963).

In another example in which working memory stimuli are used, the test protocol defines a five-pointed star as an unconditional target stimulus and six-and twelve-pointed stars as working memory stimuli, with only the one of these two symbols that most recently follows a four-pointed star in the array sequence able to serve as a target stimulus. In this example, if the array is as follows (with the array sequence number in parentheses): four-pointed star (1), six-pointed star (2), eighteen-pointed star (3), eighteen-pointed star (4), eighteen-pointed star (5), twelve-pointed star (6), five-pointed star (7), eighteen-pointed star (8), eighteen-pointed star (9), six-pointed star (10), eighteen-pointed star (11), four-pointed star (12), twelve-pointed star (13), eighteen-pointed star (14), eighteen-pointed star (15), six-pointed star (16), eighteen-pointed star (17), twelve-pointed star (18), five-pointed star (19), eighteen-pointed star (20); then array numbers 2, 7, 10, 13, 18, and 19 would be defined as target stimuli.

In any of the methods of the invention, each stimulus is displayed for a certain period of time, which may be fixed or varied, preferably randomly with durations of from 100 to 400 milliseconds. Varying the duration of stimuli presentation introduces another level of complexity to the test over previously described methods and introduces an increased memory demand on the subject. If the stimuli are visible, they can appear at fixed locations or, if another level of test complexity is desired, can be presented at random locations of the visualization device (e.g., a computer screen). Similarly, if the stimuli are audible, the sound stimuli used in the array can be generated from one location or from multiple locations. After their presentation, stimuli can then be removed, with an intervening interval of time passing (the interstimulus interval), preferably from 500 to 5,000 milliseconds (or from 500 to as much as 6,000, 7,000, 8,000, 9,000, 10,000, or 15,000 milliseconds), when no stimuli are presented before the next stimulus in the array appears. In another embodiment of a method that includes visual stimuli, the array can include masking symbols which alter the subject's visual image of target and non-target stimuli in such a manner that, once the masking symbol has been visualized, the subject could not know what the preceding stimulus was by closing their eyes and focusing on its retinal afterimage. Typically, the masking symbol is indistinct and ambiguous but includes some features of the stimulus that it is meant to obscure. For example, an exploded star would be an acceptable mask symbol for a test that includes pointed stars as target and non-target stimuli. Preferably, masking symbols occur at array positions between the positions of other target and/or non-target stimuli and immediately follow the preceding stimulus. The mask can be for a duration of 100 to 2,000 milliseconds and can be followed by an interstimulus interval of 500 to 5,000 milliseconds when nothing is presented.

The methods of the invention may be used alone, together, or in conjunction with other well-known psychological tests for determining attention or reaction time. Testing of the subject's performance may be conducted with or without providing corrective feedback to the subject during performance of the CPT.

The following examples are to illustrate the invention; they are not meant to limit the invention in any way.

### EXAMPLE 1

Assessment of Attentional State during a Computerized Test

Twenty stimuli are presented sequentially at two second intervals, over a total duration of 30 seconds. Each of the stimuli is either a target or a non-target stimulus, chosen randomly with equal probability, and chosen independently of the other stimuli. Although the total number of symbols remains fixed at 20, the numbers of target and non-target symbols vary, but generally do not differ by more than about 7. The percentage of targets to which the subject responds is calculated, and denoted T. Similarly, the percentage of responses to non-targets is denoted N.

If a subject does not discriminate between targets and non-targets, then the two percentages T and N will be similar. A subject is defined to be “Randomly” responding if N and T differ by less than 25%, and the subject responds to most of the stimuli. Conversely, a subject is “Minimally responding” if N and T agree within 25% but the subject responds to less than half of the stimuli.

For a perfect response, Tis 100%, and N is 0%. A subject is defined to be “On Task” if Tis 95% or greater, and Nis 5% or less. An impulsive subject typically responds to target stimuli with good accuracy, but exhibits errors of commission. If T is 95% or greater and N exceeds 5% (and the subject is not Randomly responding), then the subject is defined to be “Impulsive.” A subject who misses more than 5% of the target stimuli (T is less than 95%), responds to a greater percentage of target than non-target stimuli (T is greater than N) and is not Randomly or Minimally responding, is defined as “Distracted.” Finally, a subject could respond to more non-target than target stimuli, either intentionally or through confusion. If N exceeds T by more than 25%, then the subject is defined to be “Contrary.”

Depending on the test employed, even one error of commission may be indicative of an impulsive state and one error of omission may be indicative of a distracted state in test subjects who are 12 years old or older. In addition, when a working memory component is employed in the test, one can also define correct responses to target working memory stimuli as distracted responses if they occur with a long latency (such as, for example, greater than or equal to three standard deviations from the mean correct response latency for non-working memory stimuli; see Halperin, Psychological Assessment: A Journal of Consulting and Clinical Psychology 3:603-608, 1991). Similarly, one can also define a false rapid response (such as, for example, one that is less than or equal to three standard deviations from the mean correct response latency) to a working memory target to be an impulsive error.

The above procedure is repeated using different arrays of symbols of the same length. Thus, the attention state can be recorded after each array and used to determine the amount of time spent in each state, the pattern of attention states, and the range of attention states occupied by the subject.

### OTHER EMBODIMENTS

From the foregoing description, it will be apparent that variations and modifications may be made to the invention described herein to adopt it to various usages and conditions. Such embodiments are also within the scope of the following claims.

All publications mentioned in this specification are herein incorporated by reference to the same extent as if each individual publication was specifically and individually indicated to be incorporated by reference.

What is claimed is:

1. A method of determining the attentional state of a subject comprising: a) tracking movements of said subject; b) presenting to said subject a sequence of a predetermined number of stimuli over a predetermined period of time, wherein said sequence comprises an array comprising target stimuli that vary in the duration of their presentation; c) scoring the target stimuli responded to by said subject; and d) determining the attentional state of said subject based on said movements and said scoring.

2. The method of claim 1, wherein said movements are tracked for a first time period before the presentation of said stimuli and for a second time period during the presentation of said stimuli, and wherein said movements during said first time period are compared to said movements during said second time period.

3. The method of claim 1, wherein said movements are tracked for a first time period starting at the presentation of one stimulus in said array and ending at the presentation of the next stimulus in said array, wherein said subject is instructed not to respond during said first time period; and for a second time period starting at the presentation of one stimulus in said array and ending at the presentation of the next stimulus in said array, wherein said subject is instructed to respond during said second time period, wherein said movements during said first time period are compared to said movements during said second time period.

4. (canceled)

5. (canceled)

6. The method of claim 1, wherein said duration of stimuli presentation is between 100 milliseconds and 400 milliseconds.

7. The method of claim 1, wherein said stimuli are visual symbols.

8. (canceled)

9. The method of claim 7, wherein said visual symbols are presented at different locations on a screen.

10. The method of claim 7, wherein said method further comprises masking symbols in said array, wherein each of said masking symbols suppresses the afterimage created on the retina of said subject by the stimulus that precedes each of said masking symbols in said array.

11. The method of claim 10, wherein each of said masking symbols is presented for a time interval of from 100 to 2000 milliseconds.

12. The method of claim 1, wherein said stimuli are audio sounds.

13. The method of claim 12, wherein said sounds are presented at different locations in a room.

14. (canceled)

15. (canceled)

16. The method of claim 1, wherein said method further comprises scoring the target stimuli not responded to by said subject.

17. The method of claim 1, wherein said method further comprises scoring the non-target stimuli responded to by said subject.

18. The method of claim 1, wherein said method further comprises scoring the target stimuli not responded to by said subject and scoring the non-target stimuli responded to by said subject.

19. The method of claim 1, wherein said method is used to diagnose a psychological or behavioral disorder.

20. The method of claim 19, wherein said disorder is depression, an anxiety disorder, schizophrenia, drug addiction, an eating disorder, attention deficit disorder, attention deficit hyperactivity disorder, hyperkinetic disorder, a learning disorder, Alzheimer's disease, dementia, epilepsy, stroke, or traumatic brain injury.

21. (canceled)

22. The method of claim 20, wherein said disorder is attention deficit disorder.

23. (canceled)

24. (canceled)

25. (canceled)

26. (canceled)

27. A method of determining the attentional state of a subject comprising: tracking the movements of said subject; presenting to said subject a sequence of a predetermined number of stimuli over a predetermined period of time, wherein said sequence comprises an array comprising unconditional target stimuli and working memory stimuli that can function as target stimuli; scoring the target stimuli responded to by said subject; and determining the attentional state of said subject based on an evaluation of said subject's movements and said scoring of target stimuli responded to.

28. The method of claim 27, wherein said movements are tracked for a first time period before the presentation of said stimuli and for a second time period during the presentation of said stimuli, wherein said evaluation is based on a comparison of said movements during said first time period and during said second time period.

29. The method of claim 27, wherein said movements are tracked for a first time period starting at the presentation of one stimulus in said array and ending at the presentation of the next stimulus in said array, wherein said subject does not respond during said first time period; and for a second time period starting at the presentation of one stimulus in said array and ending at the presentation of the next stimulus in said array, wherein said subject responds during said second time period, wherein said evaluation is based on a comparison of said movements during said first time period and during said second time period.

30. The method of claim 27, wherein said method further comprises scoring the number of said working memory stimuli that function as target stimuli responded to by said subject.

31. (canceled)

32. (canceled)

33. (canceled)

34. The method of claim 27, wherein said method further comprises measuring a first time period required for said subject to respond to one of said unconditional target stimuli and a second time period required for said subject to respond to one of said working memory stimuli, wherein said scoring is based on a comparison of said first time period and said second time period.

35. The method of claim 27, wherein said working memory stimuli function as said target stimuli based on a predetermined positional relationship in said array.

36. The method of claim 27, wherein said stimuli are visual symbols.

37. (canceled)

38. The method of claim 36, wherein said method further comprises masking symbols in said array, wherein each of said masking symbols suppresses the afterimage created on the retina of said subject by the stimulus that precedes each of said masking symbols in said array.

39. The method of claim 38, wherein each of said masking symbols is presented for a time interval of from 500 to 2000 milliseconds.

40. The method of claim 27, wherein said stimuli are audio sounds.

41. The method of claim 40, wherein said sounds are presented at different locations in a room.

42. (canceled)

43. (canceled)

44. The method claim 27, wherein each of said target stimuli is presented for a time interval of between 100 milliseconds and 400 milliseconds.

45. The method of claim 44, wherein the duration of said time interval varies over said predetermined period of time.

46. (canceled)

47. (canceled)

48. The method of claim 27, wherein said method is used to diagnose a psychological or behavioral disorder.

49. The method of claim 48, wherein said disorder is depression, an anxiety disorder, schizophrenia, drug addiction, an eating disorder, attention deficit disorder, attention deficit hyperactivity disorder, hyperkinetic disorder, a learning disorder, Alzheimer's disease, dementia, epilepsy, stroke, or traumatic brain injury.

50. (canceled)

51. The method of claim 49, wherein said disorder is attention deficit disorder.

52. (canceled)

53. (canceled)

54. (canceled)

55. A method of determining the effects of a therapy on the attentional state of a subject, said method comprising the steps of:
a) tracking the movements of said subject; presenting to said subject a sequence of a predetermined number of stimuli over a predetermined period of time, wherein said sequence is an array comprising unconditional target stimuli and working memory stimuli; scoring the target stimuli responded to by said subject; determining the attentional state of said subject based on an evaluation of said movements and said scoring;
b) administering said therapy; and
c) repeating steps a) and b), wherein a difference in said attentional state before and after step b) indicates that said therapy has an effect on the attentional state of said subject.

56. The method of claim 55, wherein said method further comprises scoring the number of said working memory stimuli that function as target responded to by said subject in step a).

57. (canceled)

58. (canceled)

59. The method of claim 55, wherein said method further comprises scoring the target stimuli not responded to by said subject and scoring the non-target stimuli responded to by said subject.

60. The method of claim 55, wherein said working memory stimuli function as said target stimuli based on a predetermined positional relationship in said array.

61. (canceled)

62. (canceled)

63. (canceled)

64. (canceled)

65. (canceled)

66. The method claim 55, wherein each of said target stimuli is presented for a time interval of between 100 milliseconds and 400 milliseconds.

67. The method of claim 66, wherein the duration of said time interval varies over said predetermined period of time.

68. The method claim 55, wherein said method further comprises masking symbols in said array, wherein each of said masking symbols suppresses the afterimage created on the retina of said subject by the stimulus that precedes each of said masking symbols in said array.

69. The method of claim 68, wherein each of said masking symbols is presented for a time interval of from 500 to 2000 milliseconds.

70. The method of claim 69, wherein the duration of said time interval varies over said predetermined period of time.

71. (canceled)

72. (canceled)

73. (canceled)

74. (canceled)

75. (canceled)

76. (canceled)

77. (canceled)

78. The method of claim 55, wherein said method is used to determine the preferred therapy for the treatment of a psychological, neurological, or behavioral disorder.

79. (canceled)

80. (canceled)

81. (canceled)
